(19) United States
(12) Patent Application Publication
(10) Pub. No.: US 2024/0212833 A1
(43) Pub. Date: Jun. 27, 2024

(54) SYSTEM AND METHOD FOR COLLECTING FEEDBACK ON VISITS TO HEALTHCARE SERVICES FACILITIES

(71) Applicant: Amaze PBC, Longmont, CO (US)

(72) Inventor: David Mark Silverstein, Longmont, CO (US)

(21) Appl. No.: 18/088,614

(22) Filed: Dec. 25, 2022

(51) Int. Cl. G16H 40/20; H04W 4/021

(52) U.S. Cl. CPC G16H 40/20; H04W 4/021

(57) ABSTRACT

A method for providing notifications and communication to a patient based on a scheduled appointment to a healthcare services facility includes determining, using an appointment reader module, information related to a scheduled appointment to a healthcare services facility on a mobile device associated with the patient. This information includes the time and date and location of the scheduled appointment. The method further includes determining that the time and date of the scheduled appointment has been reached and the mobile device has crossed or entered a geofence area corresponding to the location of the healthcare services facility, sending a first notification to or retrieving the first notification from the mobile device, wherein the first notification requests feedback from the patient about the scheduled appointment visit, and outputting the first notification to the mobile device when the location data of the mobile device is determined to be outside of the geofenced area.

### FIELD

This application relates to a system and method for collecting feedback/ratings on visits to healthcare services facilities.

### BACKGROUND

After a health-related appointment at a healthcare services facility, a patient may want to provide feedback about the appointment. Such feedback is often desirable to healthcare providers to find ways to improve their healthcare services and general services regarding the appointment. However, most healthcare provider feedback systems do not add any value for the patient. Often the patient does not want to take the time and effort to provide feedback about his appointment. Usually, the feedback provided are complaints and so little feedback so as not to have a statistical value to the provider. Hence, it is the object of the present invention to provide a feedback system and method that is more beneficial to the healthcare provider.

### SUMMARY

This application addresses the above-mentioned desires. In one aspect of this application, a computer-implemented method for providing notifications and communication to a patient based on a scheduled appointment to a healthcare services facility is provided. The method includes the following operations performed by at least one computer processor. These operations include determining, using an appointment reader module, information related to a scheduled appointment to a healthcare services facility on a mobile device associated with the patient, wherein the information related to the scheduled appointment to the healthcare services facility includes the time and date and location of the scheduled appointment, determining that the time and date of the scheduled appointment has been reached and the mobile device associated with the patient has crossed or entered a geofence area corresponding to the location of the healthcare services facility for the scheduled appointment, sending a first notification to or retrieving the first notification from the mobile device, wherein the first notification requests feedback from the patient about the scheduled appointment visit of the patient with the healthcare services facility, and outputting the first notification to the mobile device when the location data of the mobile device is determined to be outside of the geofenced area.

In another aspect of the present application, a non-transitory computer-readable storage medium storing executable instructions is provided that, as a result of being executed by one or more processors of a computer system, cause the computer system to at least determine, using an appointment reader module, information related to a scheduled appointment to a healthcare services facility on a mobile device associated with the patient, wherein the information related to the scheduled appointment to the healthcare services facility includes the time and date and location of the scheduled appointment, determine that the time and date of the scheduled appointment has been reached and the mobile device associated with the patient has crossed or entered a geofence area corresponding to the location of the healthcare services facility for the scheduled appointment, send a first notification to or retrieving the first notification from the mobile device, wherein the first notification requests feedback from the patient about the scheduled appointment visit of the patient with the healthcare services facility, and output the first notification to the mobile device when the location data of the mobile device is determined to be outside of the geofenced area.

Further embodiments of the disclosed system and method for collecting feedback/ratings on visits to healthcare services facilities will become apparent from the following detailed description, the accompanying drawings and the appended claims.

### BRIEF DESCRIPTION OF THE DRAWINGS

FIG. 1 is a block diagram of the components of the system according to one embodiment of the present invention;

FIG. 2 is a block diagram of the client device and related elements according to the system of FIG. 1;

FIG. 3 is a flow diagram of an exemplary method according to FIG. 1;

FIG. 4 is a schematic view of a healthcare services facility surrounded by a geofence area associated with a healthcare facility and illustrating a mobile device located within the geofenced area according to the present invention;

FIG. 5 is a schematic view of a healthcare services facility with a first group of geofenced areas associated with a healthcare facility and illustrating a mobile device located within one of the geofenced areas according to the present invention;

FIG. 6 is a schematic front view of the mobile device displaying a message and “Yes” and “No” reply buttons on the display of the mobile device of the system according to FIG. 1;

FIG. 7 is a schematic front view of the mobile device displaying another message on the display of the mobile device of the system according to FIG. 1;

FIG. 8 is a schematic front view of the mobile device displaying another message and a reply message by the user on the display of the mobile device of the system according to FIG. 1;

FIG. 9 is a schematic front view of the mobile device displaying another message and a message button on the display of the mobile device of the system according to FIG. 1;

FIG. 10 is a schematic front view of the mobile device displaying an image of a video call initiated by the user on the mobile device of the system according to FIG. 1;

FIG. 11 is a schematic front view of the mobile device displaying another message and reply message on the display of the mobile device of the system according to FIG. 1; and

FIG. 12 is a schematic front view of the mobile device displaying a message requesting feedback and an area for inputting the feedback on the display of the mobile device of the system according to FIG. 1.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

As used herein, the terms “component” and “system” are intended to encompass hardware, software, or a combination of hardware and software. Thus, for example, a system or component may be a process, a process executing on a processor, or a processor. Additionally, a component or system may be localized on a single device or distributed across several devices.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

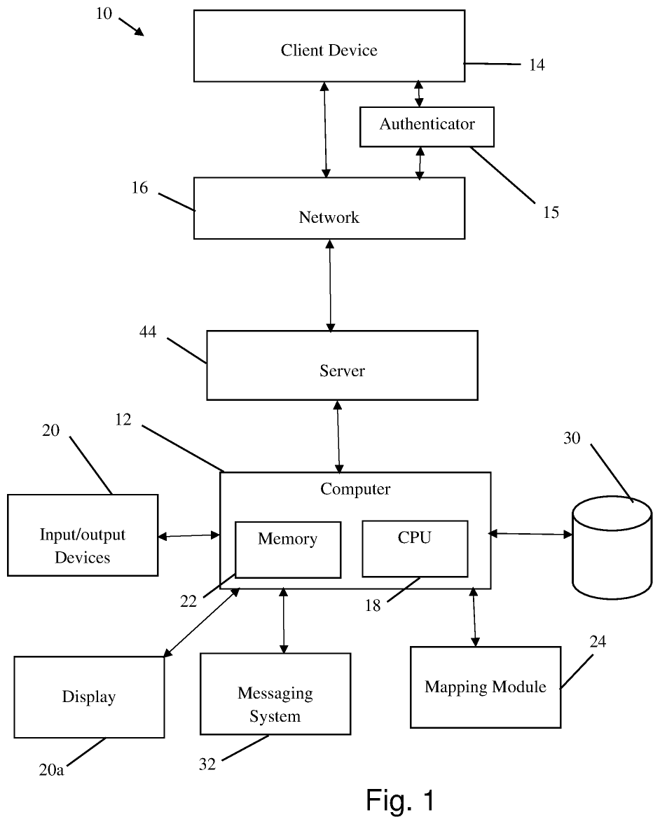

FIG. 1 shows a block diagram of a feedback/ratings system 10 that collects feedback/ratings on appointments and/or visits to healthcare services facilities and medical providers that uses multiple sources of data to unobtrusively ask for feedback only when certain conditions are met. The system 10 may include a computer 12 and a client device 14 such as a mobile device. The components may each be connected and placed in communication with one another over a computer network 16. Embodiments of the network 16 may be constructed using wired or wireless connections between each hardware component connected to the network 16.

The computer 12 may generally comprise a processor 18, otherwise referred to as a central processing unit (CPU), input/output devices 20 such as a display 20a, keyboard, printer etc. coupled to the processor 18, and memory device 22. The processor 18 may perform computations and control the functions of the computer 12, including executing instructions included in the computer code for tools and programs for creating geofenced areas and triggering a geofence notification, in the manner prescribed by the embodiments of the disclosure using the components, wherein the instructions of the computer code may be executed by the processor 18 via memory device 22. The computer code may include software or program instructions that may implement one or more algorithms for implementing the methods for providing a geofence notification. The processor 18 executes the computer code. The processor 18 may include a single processing unit, or may be distributed across one or more processing units in one or more locations (e.g., on a client and server).

The memory device 22 may include input data. The input data includes any inputs required by the computer code. The display 20a displays output from the computer code. The memory device 22 may be used as a computer usable storage medium (or program storage device) having a computer readable program embodied therein and/or having other data stored therein, wherein the computer readable program comprises the computer. The computer 12 may be accessed by a healthcare or medical professional such as a doctor, physician assistant, nurse practitioner, or other medically or non-medically trained services provider who may provide information, service, or support to the patient.

The system 10 may include a messaging system 32. The messaging system 32 may perform the functions, tasks and services of the system 10 directed toward creating notifications or messages and notification events associated with the system such as notifications to the client device 14. The message system 32 may send one or more notifications upon the occurrence of one or more of the programmed transitions. For example, when a client device 14 breaches a virtual boundary of a geofenced area (entering or exiting), a geofence notification may be transmitted or outputted to the client device 14. The message system 32 may be a combination of hardware and software resources, and may be designed to implement or execute one or more particular functions, tasks or routines of the system. The message system 32 may include self-contained components such as chipsets, specialized circuitry and one or more memory devices, and/or may be part of a program code or linked to program code containing specific programmed instructions loaded in a memory device.

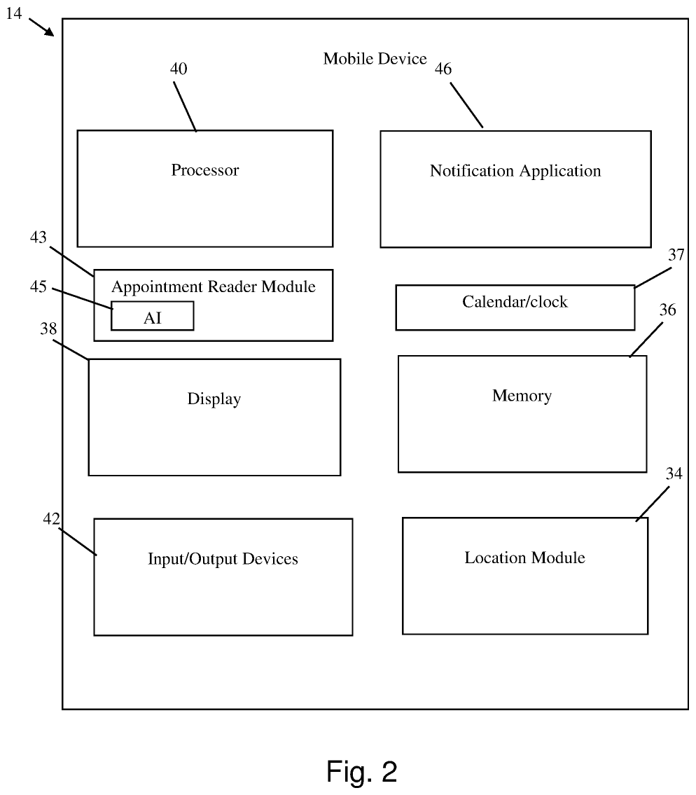

The system 10 may include an appointment reader module 43 (FIG. 2) that determines that a user member has a scheduled healthcare appointment with a healthcare services facility on the client device 14 associated with the user. The user may be the patient for the health care appointment. The term “module” may refer to a hardware based module, software based module or a module may be a combination of hardware and software resources. A module (whether hardware, software, or a combination thereof) may be designed to implement or execute one or more particular functions, tasks or routines of the system. Embodiments of hardware based modules may include self-contained components such as chipsets, specialized circuitry and one or more memory devices. A software-based module may be part of a program code or linked to program code containing specific programmed instructions loaded in a memory device.

The appointment reader module 43 may be in a form of an application that resides on the client device 14. The appointment reader modules 43 includes an artificial intelligence system (AI) 45 that is located therein. Healthcare appointments that are placed on a calendar/clock 37 of the client device 14 or on a calendar of another device such as the computer 12 that is synched with the client device 14 are analyzed by the appointment reader module 43. If the appointment reader module 43 determines that there is a scheduled healthcare appointment, the message system 32 sends a message to the client device 14 of the user that ask the user if they would like to leave feedback. If the user says no or otherwise indicates that they do not want to leave feedback, then no further messages to request feedback are sent to the client device 14. If the user replies yes or otherwise indicates that they want to leave feedback, the message system 32 will send one or more further messages to request feedback.

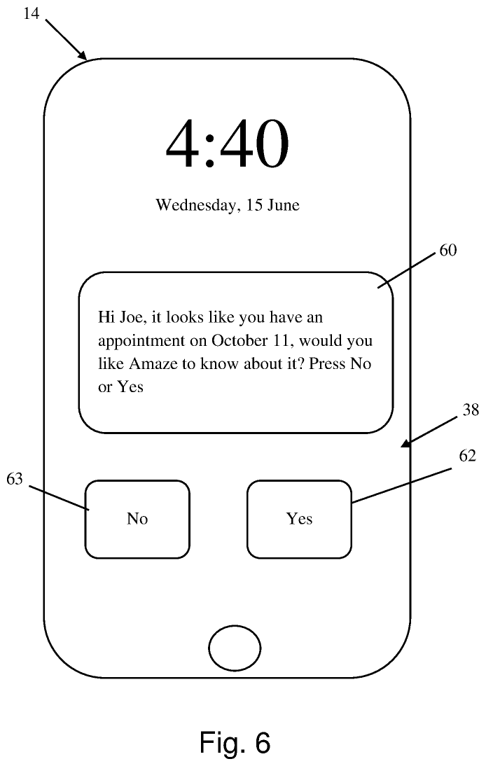

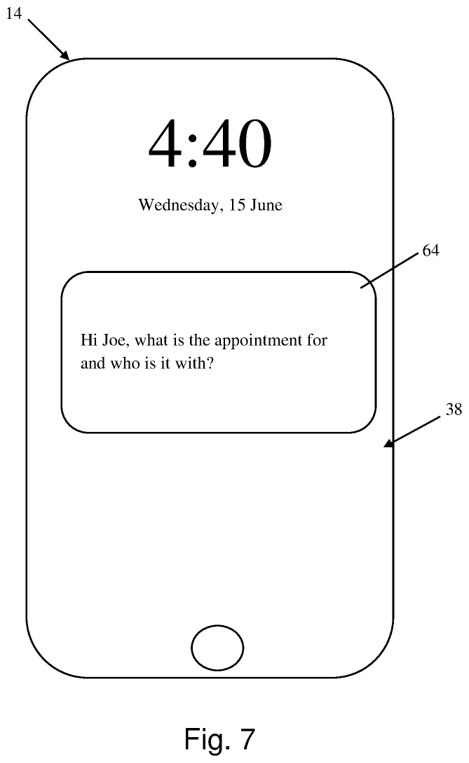

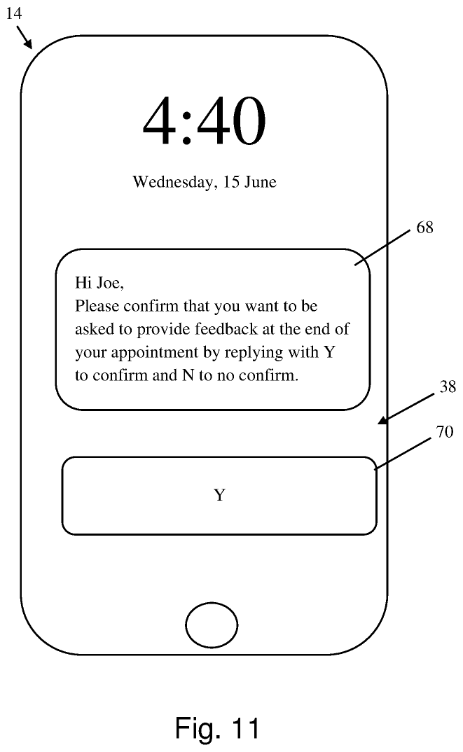

For example, as illustrated in FIG. 6, the first message 60 may say “Hey Joe, it looks like you might have made a doctor's appointment on October 11. Would you like to let Amaze know about it?” If joe says, “No.” by, for example pressing a touch button 63 displayed on a display 38 of the mobile device 14, then no further messages are sent. If Joe says “Yes”, by, for example pressing a touch button 62 displayed on a display 38 of the mobile device 14, the message system then sends another message 64 (FIG. 7) that ask Joe a few questions such as “what's the appointment for.” and “Who's it with.” The system 10 can then search for the person or entity that the user answered from databases such as national NPI database. The message system 32 may send an offer help message 65 that asks, “is there anything we can help you with?” Joe may provide answers such as for example, “I'd like to find someone to see sooner,” or “closer” or “please send me info about this medical condition,” or “I'd like to talk to an Amaze doctor before my appointment.” Joe may also answer after being prompted by a message 68 (FIG. 11) asking him to confirm that he wants to be asked for feedback.

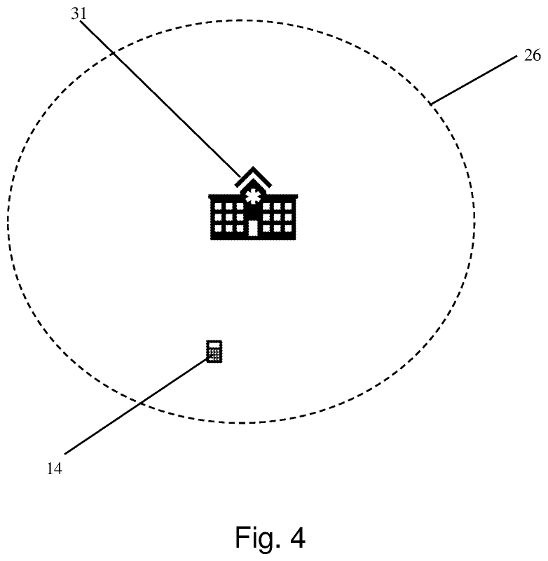

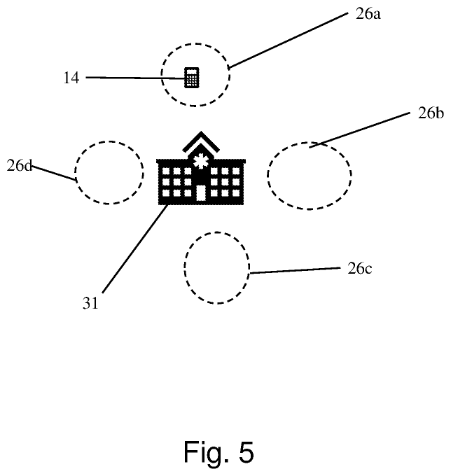

The system 10 may include a mapping module 24. The mapping module 24 may create one or more geofenced areas such as one geofenced area 26 (FIG. 4) associated with a healthcare services facility 31 based on data in the system. The geofenced area 26 may also be associated with the mobile device 14. The mapping module 24 may alternatively create a first group of geofenced areas 26a-26d (FIG. 5) based on data in the system. The first group of geofenced areas 26a-26d may be created to correspond to or associate with the healthcare services facility 31 as shown in FIG. 5 and with the mobile device 14. Each geofenced area of the first group may be entered or exited from directions or ways that differ from each of the other one or more geofenced areas in the first group of geofenced areas 26a-26d. For example, a large supermarket, or a hospital emergency room on a hospital campus may have several geofenced areas associated with it, since one large geofenced area encircling the emergency room would not work, because there may be too many false triggers for people going to the hospital for other purposes. Further, there may be cases where there are one or more parking lots, driveways or entrances that are exclusively for the emergency room, so these parking lots and other such location would be associated with a geofenced area for the emergency room. This would also allow for an earlier detection and notification compared with just having a small geofenced area around the emergency room.

In another example, a large supermarket campus may have several geofenced areas associated with it, since one large geofenced area encircling the location providing the healthcare services room would not work, because there may be too many false triggers for people going to or exiting the supermarket for other purposes. Further, there may be cases where there are one or more parking lots, driveways or entrances that are exclusively for the location providing the healthcare services, so these parking lots and other such location would be associated with a geofenced area. Additionally, the geofenced area, which may be defined by other location services such as wifi hotspots or beacons, may be inside a building versus outside. For example, the geofenced area could be a particular store or clinic inside a shopping mall or a clinic inside of a department store.

The mapping module 24 may receive geofence configuration data defining the properties of each geofenced area. The geofence configuration data may include data defining each geofences' name, location, and size or virtual boundary limits (i.e. longitude, latitude and radius, proximity, or detection of other location determining digital signals). The geofencing configured may comprise a defined geographic boundary area (a radius around an address, geo position coordinates, or other specified location or a geometric boundary such as a geofence or a proximity (distance) from a specific location.

The message system 32 may create interruption conditions based on data in the system 10 to prevent outputting messages to the client device 14. This avoids unnecessary triggers. One interruption condition may be if it is determined that the patient has requested not to send a message to provide feedback to the client device 14. The patient may set this up in the profile settings of the patient account. For example, the patient may click on a dial button or slide a slide button to enable this feature that is located next to a phrase that says, “Never ask me for feedback”. Alternatively, the patient may have requested that the message be sent to provided feedback based on certain conditions. For example, the patient may request that the messaging system 32 only send a message to request feedback if the system 10 determines from the data of the number of the patient's appointments stored in the data store that this is the patient's initial visit. The patient may set this up in the profile settings of the patient account. For example, the patient may click on a dial button or slide a slide button to enable this feature that is located next to a phrase that says “Only ask me for feedback for an initial visit”.

The patient may also send a request to the system 10 once a certain appointment is made to have the message system 32 not send a message requesting feedback about this specific appointment. For prior or subsequent appointments from this specific appointment, the message system is enabled to send message requesting feedback to the patient about the respective appointment. For example, the patient may type, voice, or otherwise input and send a message to the system 10 that says “don't ask me for feedback about this appointment” when the patient sets up the appointment or after setting up the appointment. The patient may type, voice, or otherwise input and send a message to the system that says “feel free to request feedback after this appointment” when the patient sets up the appointment or after setting up the appointment.

The system 10 may have the appointment reader module 43 via the AI 45 read the message and determine that the request for feedback not be sent for this appointment and send a control signal or otherwise communicate to the message system 32 to not send a message to the patient to request feedback about the appointment. In one example, there may be a touchscreen button displayed on the touch screen of the mobile device 14 that the patient may press to have the message system 32 not send a message to the patient to request feedback about the appointment. In another example, the patient may type, voice, or otherwise input a particular code such as “STOP” to have the message system 32 not send a message to the patient to request feedback about the appointment. If the patient has requested not to send a message to provide feedback to the client device on all of its appointments, but then decides that he or she would like to have the message system 32 send a message to request feedback for one or more particular appointments, there may be a touchscreen button displayed on the touch screen of the mobile device that the patient may press to have the message system send a message to the patient to request feedback about the appointment.

The system 10 may further comprise a location module 34 associated with the client device 14 as illustrated in FIG. 2. The location module 34 detects, processes and communicates the location of the client device 14 associated with the user. Location sensing technology may include but is limited to global positioning systems (GPS), Wi-Fi, Bluetooth, 3G, 4G, 5G, 6G, 7G cellular technology, near field communications, radio frequency identification (RFID), beacons, and any other location identifying signal. The location module 34 may be comprised of hardware and/or software capable of utilizing a positioning system to pinpoint the current location of the client device 14 and/or previously stored locations of the client device 14 that may be saved in a memory device 36 or data store 30. The location module 34 may include a transmitter, receiver and/or transceiver for receiving location data from a positioning system or broadcasting the location data to the system 10. The location module 34 may save, store and update one or more sets of location data to a memory device onboard the location module 34 or, the location module 34 may store the location information to the memory device 36 or the data store 30. The location module 34 may include any sort of system that informs the mobile device of its geolocation including, but not limited to, the Global Positioning System of satellites circling the Earth.

With continued reference to FIG. 2, the client device 14 may be a portable device such as the mobile device as previously mentioned. The mobile device 14 may optionally be any computing device small enough to hold and operate in the hand. The mobile device 14 may also be built into a vehicle. The mobile device 14 may comprise the display 38 having a flat or curved screen interface that provides a touchscreen interface with digital buttons and keyboard, and/or physical buttons along with a physical keyboard. The mobile device 14 may be voice activated. The mobile device 14 may connect to the internet and interconnect with other devices such as car entertainment systems or headsets via Wi-Fi, Bluetooth, cellular networks or near field communication (NFC). The flat screen interface may be an LCD flat screen interface, an OLED flatscreen interface, or other suitable type of flat screen interface. Alternatively, the display 38 may being the form of a hologram. The mobile device 14 may be a cell phone, smart phone, smart watch, tablet, PDA, laptop, notebook or other suitable portable or mobile device. The mobile device 14 is configured to detect its location and hence the location of a user using the mobile device 14 or other person near the mobile device 14.

The mobile device 14 includes one or more processors 40 and the memory device 36. The memory device 36 may contain a user identification module that may in turn contain a user identifier and/or user information. The user identifier may be a unique number or code that uniquely identifies the user of the mobile device. The user identifier may be used store to be a member of the system. The system 10 includes an authenticator 15 to check the user identifier to see if the user is a member in the system 10.

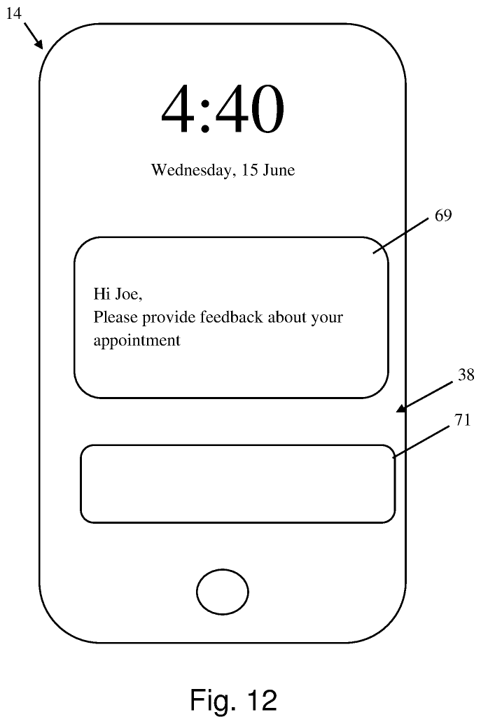

The mobile device 14 may also include input/output devices 42 such as a camera capable of taking still or video pictures and have the capability to make video calls (see FIG. 5). An antenna in the mobile device may send and receive wireless signals from sources such as the radio antenna and satellite. The antenna may, in some implementations, communicate directly with the server such as by exchanging wireless signals. The mobile device 14 may further comprise other input/output devices 42, such as a microphone and a speaker used, for example, in an implementation in which the mobile device 14 functions as a telephone. In some implementations, the mobile device 14 may also include a calendar/clock 37 and a network interface. The calendar/clock 37 may calculate time, date, and other data that can be derived from time data and date data. This time and date data may be based on GPS signals continually received by the mobile device. The message system 32 may receive the time and date data from the calendar/clock 37 to determine when to send messages and what type of message or notification to send. For example, the message system 32 may send the message 69 (FIG. 12) to request feedback when the appointment time has ended or a predetermine time after the appointment time as ended. The message system 32 may send the message to request feedback based on when the client device has exited the geofence area associated with the healthcare services facility of the appointment after the appointment has ended.

The mobile device 14 includes applications that manage interactions between a server 44 (FIG. 1) and the mobile device 14. The applications may include a notification application 46. The data store 30 associated with the system 10 may contain data on healthcare services facilities and their type. For example, the type of healthcare services facility stored in the data store 30 may be a hospital, free-standing emergency, urgent care, or walk-in clinic. One or more of the healthcare services facilities stored in the data store 30 may be associated with the patient associated with the mobile device 14. For example, the healthcare services facility may be in the patient's insurance network with this data stored in the data store 30.

The data store 30 may also store personal and medical information about the patient in the form of a record. This and other information may be made available to the patient or other person via the mobile device 14 or computer 12. The data store 30 may store an address associated with a property and a geofenced area associated with the property. The data store 30 also may store all the mobile phone numbers of the smartphones which have the notification application 46 installed. The installed notification application 46 has the geofence information so that the mobile device 14 knows, using GPS technology, whether it is inside or outside the geofenced area.

The data store 30 may store information on patient(s) or person(s) associated with the mobile device related to their medical history to speed the process of receiving the services of the healthcare facility, lists of medical questions to ask, ratings on the facility being entered or the medical professionals practicing at that facility, information about whether the facility and\or medical providers participate in their insurance network, the medical providers practicing at the healthcare facility, wait times at the healthcare facility, and the proximity to other healthcare facilities and information about the prices of the facility or providers. This data may be displayed on the display 38 of the mobile device 14. The data store 30 may store the interruption conditions for preventing the outputting of the notification when the location data of the mobile device is determined to exit the geofenced area that are created using the mapping module 24 in exemplary embodiments.

The data store 30 may store information on the medical history of the patient, the insurance network of the patient, where the patient lives and works, the price information of the healthcare service being performed for the patient, preferences of the patient, the knowledge and skill and self-confidence of the patient to manage healthcare, and the location of the healthcare services facility and patient and a geofenced area indicating that the patient is in close proximity to the healthcare services facility when entered into by the mobile device associated with the patient.

The computer 12 and mobile device 14 may communicate with the server 44 via the internet over the network 16 as illustrated in FIG. 1. The network may include any one or combination of multiple different types of networks, such as cable networks, local area networks, personal area networks, wide area networks, the Internet, wireless networks, ad hoc networks, mesh networks, and/or the like. In some implementations the satellite and/or the radio antenna may provide network connectivity to the mobile device as well as provide geolocation. For example, the radio antenna may provide network access to the mobile device according to the International Mobile Telecommunications-2000 standards (3G network) or the International Mobile Telecommunications Advanced standards (4G network) or the 5G or 6G networks. Other implementations may include one source of geolocation data such as the satellite and a separate source of network connectivity such as a Wi-Fi hotspot. The server may house or otherwise have a connection to multiple data stores including user information and/or other data stores. The server 44 and data stores can be stored where desired, for example in a cloud.

Generally, the user information contains information about the user associated with the mobile device 14. The notification application 46 is operatively connected to the server 44 which is connected to the data store 30. The notification application 46 has notifications and messages associated with different types of conditions. For example, FIG. 12 shows a notification message 69 to request feedback generated by the messaging system 32 when the mobile device 14 exits a geofenced area associated with a healthcare facility after the user finished his or her appointment at the health care facility. The notifications or messages may be stored in the memory 36 of the mobile device 14 or in the data store 30. Alternatively, or in addition, the notification may include a voice message that may include the above-mentioned information.

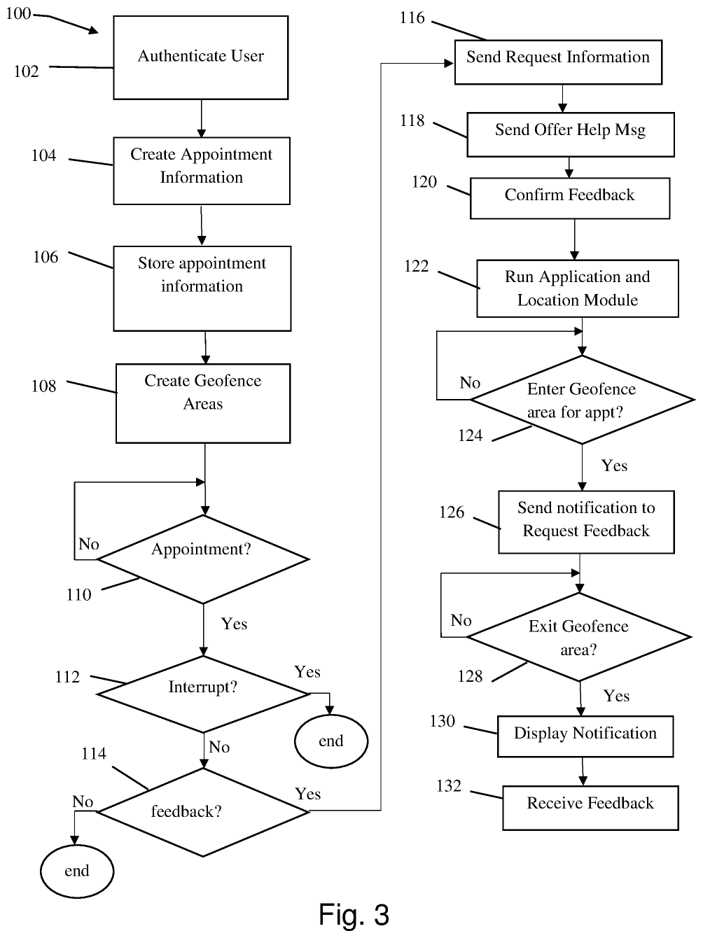

With reference now to FIG. 3, an example methodology 100 is illustrated and described. While the methodology is described as being a series of acts or steps that are performed in a sequence, it is to be understood that the methodology is not limited by the order of the sequence. For instance, some acts or steps may occur in a different order than what is described herein. In addition, a step may occur concurrently with another step. Furthermore, in some instances, not all steps may be required to implement a methodology described herein.

Moreover, the steps or acts described herein may be computer-executable instructions that can be implemented by one or more processors and/or stored on a computer-readable medium or media. The computer-executable instructions may include a routine, a sub-routine, programs, a thread of execution, and/or the like. Still further, results of acts of the methodology may be stored in a computer-readable medium, displayed on the display device, and/or the like.

In each step of this sequence of client-server message exchanges, a computer may process a request and return data. In step 102, the system authenticates via the authenticator 15 that the patient is authorized to receive messages from the system 10. In step 104, the patient's scheduled appointment information is created. The information may be typed, voiced, or otherwise inputted into the computer 12 and stored in the data store. The information includes the date, time and location of the healthcare services facility 31 associated with the appointment and the healthcare professional that the appointment is with. The patient may then put this appointment information on his or her calendar in his mobile device 14. Alternatively, the system 10 may automatically place the appointment information on the patient's calendar in the mobile device 14. Alternatively or in addition, the system may be placed on a calendar of the computer 12 or another device that is synched with the mobile device. In step 106, the information related to the scheduled appointment is stored in the data store 30.

In step 108, the geofence areas are created using the mapping module 24 based on data in the data store 30. One or more of the geofenced areas created may correspond to or be associated with the mobile device 14 associated with the patient and a location that has the healthcare services facility 31 that the patient will have his or her appointment. The facility 31 may offer one or more preventative healthcare services or one or more healthcare services. For example, one geofenced area 26 may be created for the facility 31 as shown in FIG. 4. In another example, a first group of geofenced areas 26a-26d may be created to correspond to the healthcare services facility 31 as shown in FIG. 5. For example, a location such as an emergency room or an area that screens for cancer on a hospital campus may have several geofenced areas, since one large geofenced area encircling that location would not work, because there may be too many false triggers for people going to or from the hospital for other purposes. Further, there may be cases where there are one or more parking lots, driveways or entrances that are exclusively for that location, so these parking lots and other such locations would be associated with a geofenced area for that location.

In step 110, the calendar of the patient's mobile device 14 is then analyzed by the appointment reader module 43 and a query is performed to determine if the appointment reader module 43 determines that there is a healthcare appointment. If so, the system 10 checks for interrupt conditions. As previously mentioned, the interrupt conditions may be based on data in the system 10 to prevent outputting messages to the client device 14. This avoids unnecessary triggers. One interruption condition may be if it is determined that the patient has requested not to send a message to provide feedback to the client device 14. The patient may set this up in the profile settings of the patient account. For example, the patient may click on a dial button or slide a slide button to enable this feature that is located next to a phrase that says, “Never ask me for feedback”. Alternatively, the patient may have requested that the message be sent to provided feedback based on certain conditions. For example, the patient may request that the messaging system 32 only send a message to request feedback if the system 10 determines from the data of the number of the patient's appointments stored in the data store that this is patient's initial visit. The patient may set this up in the profile settings of the patient account. For example, the patient may click on a dial button or slide a slide button to enable this feature that is located next to a phrase that says “Only ask me for feedback for an initial visit”.

The patient may also send a request to the system 10 once a certain appointment is made to have the system 10 not send a message requesting feedback about this specific appointment. For prior or subsequent appointments from this specific appointment, the message system is enabled to send message requesting feedback to the patient about the respective appointment. For example, the patient may type, voice, or otherwise input and send a message to the system that says “don't ask me for feedback about this appointment” when the patient sets up the appointment or after setting up the appointment and the patient may type, voice, or otherwise input and send a message to the system that says “feel free to request feedback after this appointment”.

If there is no interrupt condition present, in step 114, the message system 32 sends a message 60 to the mobile device 14 of the user that ask the user if they would like to leave feedback. As previously mentioned, an example message 60 may be displayed on the display 38 of the mobile device 14 that says “Hi Joe, it looks like you have an appointment on October 11, would you like Amaze to know about it?”. The user may press a touch button 62 labeled “Yes” displayed on the display 38 to indicate that they want the system 10 to know about the appointment and be asked for feedback after the appointment is over, or press a touch button 63 labeled “No” displayed on the display 38 if the user does not want the system 10 to know about the appointment and be asked for feedback after the appointment is over. If the user says no or otherwise indicates that they do not want to leave feedback, then no further messages to request feedback are sent to the mobile device 14.

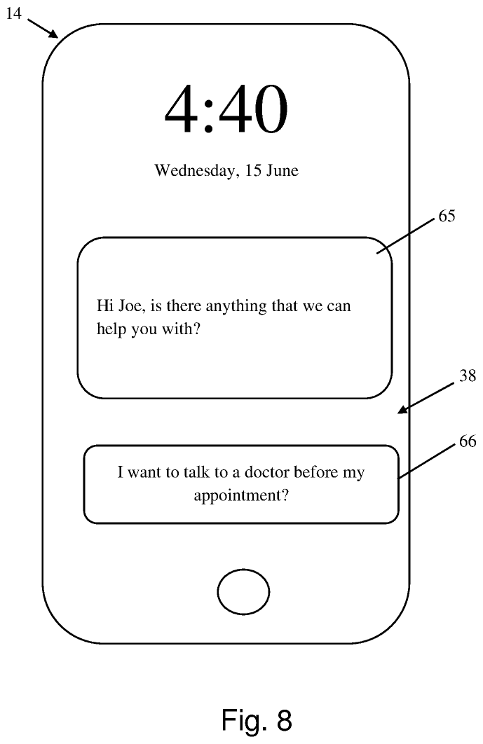

If the user replies yes or otherwise indicates that they want to leave feedback, then in step 116, the message system 32 will send one or more further messages or notifications. In particular, the message system 32 sends a message 64 that request information from the patient about the patient's appointment. The message 64 may request information about what the appointment is for and who is it with, the medical provider, the practice of the facility for the appointment (e.g. imaging). For example, the message 64 may be displayed on the display 38 of the mobile device 14 that says “Hi Joe, what is the appointment for and who is it with?”. The system 10 may perform a query or search a database for information about the entity or person that the patient provided in response to the request as to who the appointment is with. In step 118, the message system 32 may send an offer help message 65 to the patient that asks the patient if there is anything that they can help with as illustrated in FIG. 8. If the patient responds to the offer help message by sending a message to the system 10 that they want to find a doctor or other health care profession that they can see sooner for an appointment, the system 10 will perform a query to search in the database for a doctor or other healthcare professional that is available to see the patient sooner than the patient's appointment and send a message to the patient information about an available earlier appointment for the patient or that there is no earlier appointment.

If the patient responds by sending a message to the system 10 that they want to find a doctor or other health care profession that is closer to them for appointment, the system 10 will perform a query to search in the database for a doctor or other healthcare professional that is available to see the patient that is located closer to the patient's residential address, current location or other location desired by the patient than the location of the patient's appointment and send a message to the patient information about an available appointment that is closer to the patient's desired location or that there is no appointment location closer to the patient's desired location. If the patient responds to the offer help message 65 that the patient would like more information about the patient's medical condition, the system will cause the message system to send a message about more information about the patient's medical condition. If the patient responds to the offer help message 65 that the patient would like more information about the patient's medical condition, the system will cause the message system to send a message about more information about the patient's medical condition.

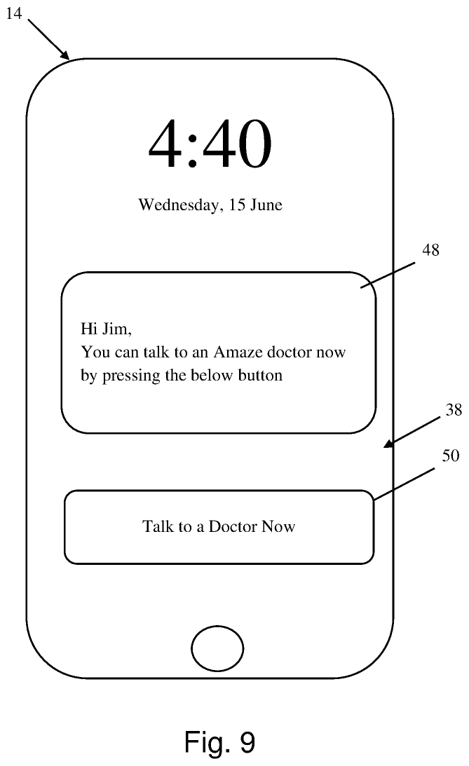

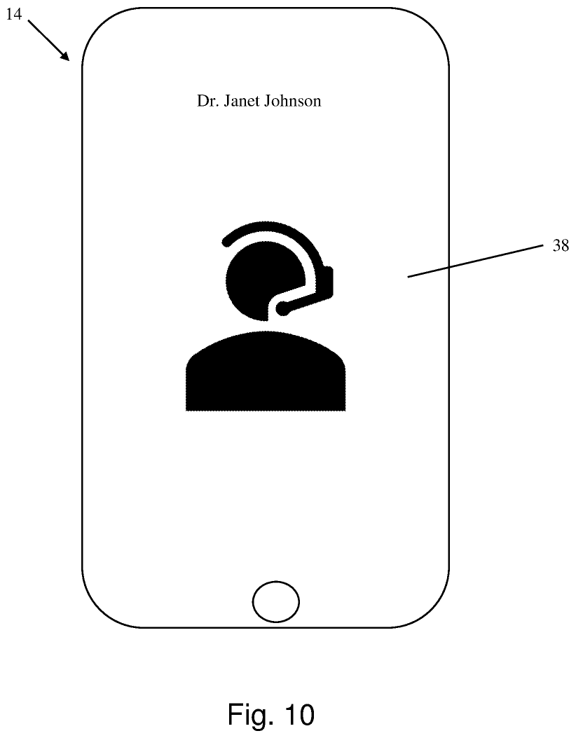

If the patient responds to the offer help message 65 with a message 66 (FIG. 8) that the patient would like to talk to a doctor before my appointment, the system 10 may send the phone number, email, and/or other contact information of a doctor who treats, diagnosis or otherwise deals with the medical condition that the patient has the appointment for. As illustrated in FIG. 9, the message system 32 may also send a message 48 and message button 50 for making video calls displayed on the display 38 of the mobile device 14 to give the patient the option to place an audio or video call with a qualified healthcare professional. The message 48 may provide the patient an invitation or suggestion to initiate a video or audio call to a qualified person that can help the patient by pressing the message button that will then promptly place the audio or video call to the qualified healthcare professional. FIG. 10 is a schematic front view of the mobile device 14 displaying an image of a video call initiated by the user on the mobile device 14 of the system 10.

In step 120, the system 10 may prompt the patient with a message 68 (FIG. 11) displayed on the display 38 of the mobile device 14 to confirm that the patient wants to be asked for feedback at the end of the patient's appointment. The message 68 may prompt the patient to confirm by sending a reply message 70 with the letter “Y” to confirm or “N” to not confirm. Alternatively, there may be a yes message button displayed on the display 38 that the patient may press to confirm or a no message button displayed on the display 38 that the patient may press to not confirm.

In step 122, the mobile device 14 is turned on with the notification application 46 launched and running and the location module 34 running so that the system 10 receives location data of the mobile device 14. In step 124 the notification application 46 determines that the mobile device 14 has crossed or enter or is otherwise located within the geofenced area associated with the healthcare services facility 31 stored in the data store 30 that the patient scheduled the appointment with. The appointment reader module 43 then checks the calendar and data store 30 to see if the patient has an appointment at that time and location.

If the mobile device 14 is located within the geofenced area and the patient has a scheduled appointment at that time and that healthcare services facility 31 at that location, then in step 126, the messaging system 32 sends the notification 69 requesting feedback to the mobile device 14 or that notification 69 requesting feedback is retrieved from the memory 36 of the mobile device 14. In step 128, determines whether the mobile device 14 has exited or is outside the geofence area 26 associated with the scheduled appointment based on the location data of the mobile device 14. In step 130, the notification 69 is displayed on the display 38 of the mobile device 14 when the notification application 46 determines based on the location data of the mobile device 14 that the mobile device 14 has exited or is outside the geofence area 26 associated with the scheduled appointment. This may mean that the scheduled appointment is over and the patient has departed. The notification 69 may include series of feedback questions for the patient to answer and/or an area on the display 38 for the patient to leave feedback by typing voicing or otherwise inputting the feedback using his or her mobile device 14. Alternatively or in addition, other ways to output the message or notification 69 may be provided such as an audio message outputted through the speakers of the mobile device 14. In step 132, the system 10 receives the feedback from the patient, who typed, voiced or otherwise inputted the feedback using his or her mobile device 14. After the patient is finished answering the question or leaving feedback in the area 71 on the display, the feedback is stored in the data store 30 to be accessed by the system. If there is an interruption condition, no notification requesting feedback is outputted to the mobile device 14 and the process ends. Alternatively, or in addition, if there is an interruption condition, the messaging system 32 does not send the notification 69 requesting feedback to the mobile device 14 or that the notification 69 requesting feedback is not retrieved from the memory 36 of the mobile device 14.

The system and method for collecting feedback/ratings on visits to healthcare services facilities enables the collection of feedbacks on visits by the patients to the healthcare services facilities in an unobtrusive manner to the patients when certain conditions are met. Although various embodiments of the disclosed system and method for collecting feedback/ratings on visits to healthcare services facilities have been shown and described, modifications may occur to those skilled in the art upon reading the specification. The present application includes such modifications and is limited only by the scope of the claims.

What is claimed is:

1. A computer-implemented method for providing notifications and communication to a patient based on a scheduled appointment to a healthcare services facility, the method comprising the following operations performed by at least one computer processor:
determining, using an appointment reader module, information related to a scheduled appointment to a healthcare services facility on a mobile device associated with the patient, wherein the information related to the scheduled appointment to the healthcare services facility includes the time and date and location of the scheduled appointment;
determining that the time and date of the scheduled appointment has been reached and the mobile device associated with the patient has crossed or entered a geofence area corresponding to the location of the healthcare services facility for the scheduled appointment;
sending a first notification to or retrieving the first notification from the mobile device, wherein the first notification requests feedback from the patient about the scheduled appointment visit of the patient with the healthcare services facility; and
outputting the first notification to the mobile device when the location data of the mobile device is determined to be outside of the geofenced area.

2. The computer-implemented method of claim 1 further comprising:
prior to determining that the time and date of the scheduled appointment has been reached and the mobile device associated with the patient has crossed or entered a geofence area corresponding to the location of the healthcare services facility for the scheduled appointment, outputting a second notification to the mobile device, wherein the second notification asks the patient if the patient wants the first notification on their mobile device outputted to the mobile device.

3. The computer-implemented method of claim 2 further comprising receiving a response from the mobile device of the patient that the patient does not want the first notification outputted on their mobile device, preventing one of or any combination of sending the first notification to or retrieving the first notification from the mobile device or outputting the first notification to the mobile device when the location data of the mobile device is determined to be outside of the geofenced area in response to receiving the response from the mobile device of the patient that the patient does not want the first notification outputted on the mobile device.

4. The computer-implemented method of claim 2 further comprising receiving a response from the mobile device of the patient that the patient wants the first notification outputted on their mobile device, outputting a third notification that asks the patient what the nature of the scheduled appointment is and who it is with.

5. The computer-implemented method of claim 4 further comprising performing a query on a database based on who the scheduled appointment is with.

6. The computer-implemented method of claim 3 further comprising outputting an offer help message to the mobile device asking the patient if the patient wants help.

7. The computer-implemented method of claim 6 further comprising receiving a response from the mobile device of the patient that the patient would like to talk to a healthcare professional, sending a fourth notification that has information about a healthcare professional for the patient to talk to and an invitation or suggestion to initiate a video or audio call to the healthcare professional by displaying a button on a display of the mobile device to be pressed to initiate the video or audio call to the healthcare professional.

8. The computer-implemented method of claim 1 further comprising determining if there are interrupt conditions that prevent one of or any combination of sending the first notification to or retrieving the first notification from the mobile device or outputting the first notification to the mobile device when the location data of the mobile device is determined to be outside of the geofenced area.

9. The computer-implemented method of claim 8 further comprising determining that this is not an initial appointment of the patient of a healthcare professional at the healthcare services facility, wherein the interrupt condition is that the scheduled appointment is not an initial appointment of the patient of the healthcare professional at the healthcare services facility.

10. The computer-implemented method of claim 8 further comprising determining that the patient has requested not to be notified to provide feedback of the scheduled appointment, wherein the interrupt condition is that that patient has requested not to be notified to provide feedback of the scheduled appointment.

11. The computer-implemented method of claim 1, wherein the information related to the scheduled appointment to the healthcare services facility includes the medical provider for the scheduled appointment.

12. The computer-implemented method of claim 1, wherein the information related to the scheduled appointment to the healthcare services facility includes the nature of the scheduled appointment.

13. The computer-implemented method of claim 1 further comprising authenticating that the patient is authorized to receive the first notification.

14. A non-transitory computer-readable storage medium storing executable instructions that, as a result of being executed by one or more processors of a computer system, cause the computer system to at least:
determine, using an appointment reader module, information related to a scheduled appointment to a healthcare services facility on a mobile device associated with the patient, wherein the information related to the scheduled appointment to the healthcare services facility includes the time and date and location of the scheduled appointment;
determine that the time and date of the scheduled appointment has been reached and the mobile device associated with the patient has crossed or entered a geofence area corresponding to the location of the healthcare services facility for the scheduled appointment;
send a first notification to or retrieving the first notification from the mobile device, wherein the first notification requests feedback from the patient about the scheduled appointment visit of the patient with the healthcare services facility; and
output the first notification to the mobile device when the location data of the mobile device is determined to be outside of the geofenced area.

15. The non-transitory computer-readable storage medium of claim 14 wherein the instructions further including instructions that, as a result of being executed by one or more processors, cause the computer system to:
prior to determine that the time and date of the scheduled appointment has been reached and the mobile device associated with the patient has crossed or entered a geofence area corresponding to the location of the healthcare services facility for the scheduled appointment, output a second notification to the mobile device, wherein the second notification asks the patient if the patient wants the first notification on their mobile device outputted to the mobile device.

16. The non-transitory computer-readable storage medium of claim 15 wherein the instructions further including instructions that, as a result of being executed by one or more processors, cause the computer system to:
receive a response from the mobile device of the patient that the patient does not want the first notification outputted on their mobile device, prevent one of or any combination of sending the first notification to or retrieving the first notification from the mobile device or outputting the first notification to the mobile device when the location data of the mobile device is determined to be outside of the geofenced are in response to receiving the response from the mobile device of the patient that the patient does not want the first notification outputted on the mobile device.

17. The non-transitory computer-readable storage medium of claim 14 wherein the instructions further including instructions that, as a result of being executed by one or more processors, cause the computer system to:
determine if there are interrupt conditions that prevent one of or any combination of sending the first notification to or retrieving the first notification from the mobile device or outputting the first notification to the mobile device when the location data of the mobile device is determined to be outside of the geofenced area.

18. The non-transitory computer-readable storage medium of claim 17 wherein the instructions further including instructions that, as a result of being executed by one or more processors, cause the computer system to:
determine that this is not an initial appointment of the patient of a healthcare professional at the healthcare services facility, wherein the interrupt condition is that the scheduled appointment is not an initial appointment of the patient of the healthcare professional at the healthcare services facility.

19. The non-transitory computer-readable storage medium of claim 14 wherein the instructions further including instructions that, as a result of being executed by one or more processors, cause the computer system to:
determine that the patient has requested not to be notified to provide feedback of the scheduled appointment, wherein the interrupt condition is that that patient has requested not to be notified to provide feedback of the scheduled appointment.

20. The non-transitory computer-readable storage medium of claim 14 wherein the instructions further including instructions that, as a result of being executed by one or more processors, cause the computer system to:
authenticate that the patient is authorized to receive the first notification.
